(19) United States
(12) Patent Application Publication
(10) Pub. No.: US 2016/0296317 A1
(43) Pub. Date: Oct. 13, 2016

(54) REDUCING STENT FOR A TRANSJUGULAR INTRAHEPATIC PORTOSYSTEMIC SHUNT

(71) Applicants: Hans Timmermans, Portland, OR (US); John Kaufman, Lake Oswego, OR (US)

(72) Inventors: Hans Timmermans, Portland, OR (US); John Kaufman, Lake Oswego, OR (US)

(73) Assignee: OREGON HEALTH & SCIENCE UNIVERSITY, PORTLAND, OR (US)

(21) Appl. No.: 15/093,521

(22) Filed: Apr. 7, 2016

Related U.S. Application Data
(60) Provisional application No. 62/145,279, filed on Apr. 9, 2015.

(51) Int. Cl. A61F 2/07; A61F 2/848; A61F 2/94; A61F 2/844

(52) U.S. Cl. CPC A61F 2/07; A61F 2/844; A61F 2/848; A61F 2/94; A61F 2002/8483; A61F 2002/068

(57) ABSTRACT

A stent used in reducing a diameter of a transjugular intrahepatic portosystemic shunt (TIPS) for secondary restriction of flow through a TIPS tract is disclosed. In one example approach, a reducing stent comprises a tube-shaped framework with a tubular material affixed around an outer surface of the framework and extending through the interior of the framework to define a reducing passage in the interior of the framework. In another example approach, a transjugular intrahepatic portosystemic shunt system including a transjugular intrahepatic portosystemic shunt and a reducing stent mounted within an interior of the transjugular intrahepatic portosystemic shunt is disclosed.

### FIELD

The present disclosure relates to a reducing stent for reducing the flow passage of a duct in a living body, such as especially a transjugular intrahepatic portosystemic shunt (TIPS).

### BACKGROUND

Transjugular intrahepatic portosystemic shunt or transjugular intrahepatic portosystemic stent shunting (commonly abbreviated as TIPS or DIPS) is an artificial channel within the liver that establishes communication between the inflow portal vein and the outflow hepatic vein. It is used to treat portal hypertension (which is often due to liver cirrhosis) which frequently leads to intestinal bleeding (esophageal varices) and the buildup of fluid within the abdomen (ascites).

A TIPS bypasses the effective vascular resistance of the liver. The result is a reduced pressure gradient across the liver and a decreased portal venous pressure. This, in turn, lessens the pressure on the blood vessels in esophagus or viscera; future bleeding is less likely to occur. Lower pressure also allows ascites to be reabsorbed although this may take weeks or months to occur.

Transjugular intrahepatic portosystemic shunts are typically placed under fluoroscopic guidance by an interventional radiologist. Access to the liver may be gained via the internal jugular vein in the neck. Once access to the jugular vein is confirmed, a guidewire and introducer sheath may be placed to facilitate the shunt's placement. This enables the interventional radiologist to gain access to the patient's hepatic vein by traveling from the superior vena cava into the inferior vena cava and finally the hepatic vein. Once the catheter is in the hepatic vein, a wedge pressure may be obtained to calculate the pressure gradient in the liver. Following this, a contrast agent or carbon dioxide may be injected to locate the portal vein. Then a special needle known as a Colapinto is advanced through the liver parenchyma to connect the hepatic vein to the large portal vein branch, near the center of the liver. The channel for the shunt may then be created by inflating an angioplasty balloon within the liver along the tract created by the needle. The shunt is then completed by placing a mesh tube known as a stent or endograft to maintain the tract between the higher pressure portal vein and the lower pressure IVC. After the procedure, fluoroscopic images may be made to show placement. Pressure measurements in the portal vein and inferior vena cava are often done.

With liver damage, especially cirrhosis of the liver, intravenous blood flow from the portal vein through the liver to the hepatic veins is reduced. An increase in blood pressure results on the portal vein side, which can result in esophageal varices, i.e. varicose veins in the area of the esophagus. If the latter leak, the danger exists of the patient bleeding to death. In some surgical approaches, a portocaval, mesocaval or splenorenal anastomosis can be performed to reduce blood pressure, i.e. to perform a connection by anastomosis of the portal venous trunk and the inferior vena cava or the intestinal vein (superior vena mesenterica) or between the plenic vein and renal vein. As a result, a reduction of pressure in the described portal hypertension takes place.

Hepatic encephalopathy (HE) is a common complication following the creation of a transjugular intrahepatic portosystemic shunt (TIPS). This condition is characterized by confusion, disorientation, obtundation, abnormal sleep patterns, and overall alterations in quality of life. New or worsened HE after TIPS placement has been reported to occur in up to 35% of patients, but conservative medical therapy often is sufficient to reverse the problem. HE that is refractory to conservative therapy develops in up to 7% of patients with a TIPS. For such patients, further intervention, including shunt reduction or occlusion and potentially emergent liver transplantation, may be required.

Various percutaneous techniques may be used to treat refractory HE to alter the hemodynamics through the shunt by occluding or reducing its diameter. Although effective in reversing HE, complete shunt occlusion, using balloons or coils, may have abrupt, life-threatening hemodynamic consequences.

### SUMMARY

Accordingly, the present disclosure is directed to a stent used in reducing a diameter of a transjugular intrahepatic portosystemic shunt (TIPS) for secondary restriction of flow through a TIPS tract. In one example approach, a stent used in reducing a diameter of a transjugular intrahepatic portosystemic shunt comprises a tube-shaped framework with a tubular material affixed around an outer surface of the framework and extending through the interior of the framework to define a reducing passage in the interior of the framework. In another example approach, a transjugular intrahepatic portosystemic shunt system including a transjugular intrahepatic portosystemic shunt and a reducing stent mounted within an interior of the transjugular intrahepatic portosystemic shunt is disclosed.

Such an approach provides a simple device to reduce flow/increase back pressure in a TIPS tract by a simple adjustable fix to control pressure gradients and/or flow across the TIPS tract. For example, by including such a reducing stent in a TIPS tract, the reducing stent may be easily dilated in response to occlusions of flow through the TIPS tract. Embodiments described herein may be used to treat complications following the creation of a transjugular intrahepatic portosystemic shunt, e.g., hepatic encephalopathy.

The above Background and Summary sections are provided to introduce a selection of concepts in a simplified form that are further described below in the Detailed Description. This Background and Summary are not intended to identify key features or essential features of the disclosed subject matter, nor are they intended to be used to limit the scope of the disclosed subject matter. Furthermore, the disclosed subject matter is not limited to implementations that solve any or all disadvantages noted in any part of this disclosure. Additionally, issues identified throughout this disclosure are not necessarily admitted to be well known and are recognized by the inventors of the present application.

### BRIEF DESCRIPTION OF THE SEVERAL VIEWS OF THE DRAWINGS

FIG. 1 shows example frameworks for a reducing stent in accordance with various embodiments.

FIG. 2 illustrates a method of making a reducing stent in accordance with various embodiments.

FIG. 3 shows an example reducing stent in accordance with the disclosure.

FIG. 4 shows another example reducing stent in accordance with the disclosure.

FIG. 5 shows example embodiments of a reducing stent in accordance with the disclosure.

FIG. 6 shows an example transjugular intrahepatic portosystemic shunt system in accordance with the disclosure.

FIG. 7 shows an illustration of a shunt between a portal vein and a hepatic vein in accordance with the disclosure.

### DETAILED DESCRIPTION

Embodiments described herein are directed to apparatuses and systems used in reducing a diameter of a transjugular intrahepatic portosystemic shunt (TIPS) for secondary restriction of flow through a TIPS tract. In the following detailed description, reference is made to the accompanying drawings which form a part hereof, and in which are shown by way of illustration embodiments that may be practiced. It is to be understood that other embodiments may be utilized and structural or logical changes may be made without departing from the scope of the disclosure. Therefore, the following detailed description is not to be taken in a limiting sense.

Embodiments described herein include a stent used in reducing a diameter of a transjugular intrahepatic portosystemic shunt (TIPS) for secondary restriction of flow through a TIPS tract. Embodiments described herein may be used for reduction of the available flow area of the shunt by inserting a reducing part and as such a reducing stent to reduce the diameter of such a duct through which liquid flows in the body, such as such a transjugular intrahepatic portosystemic shunt (TIPS).

Turning to the figures, various embodiments of a stent and transjugular intrahepatic portosystemic shunt system for reducing a diameter of a transjugular intrahepatic portosystemic shunt (TIPS) for secondary restriction of flow through a TIPS tract are shown. Like-numbered elements correspond to like elements throughout the figures.

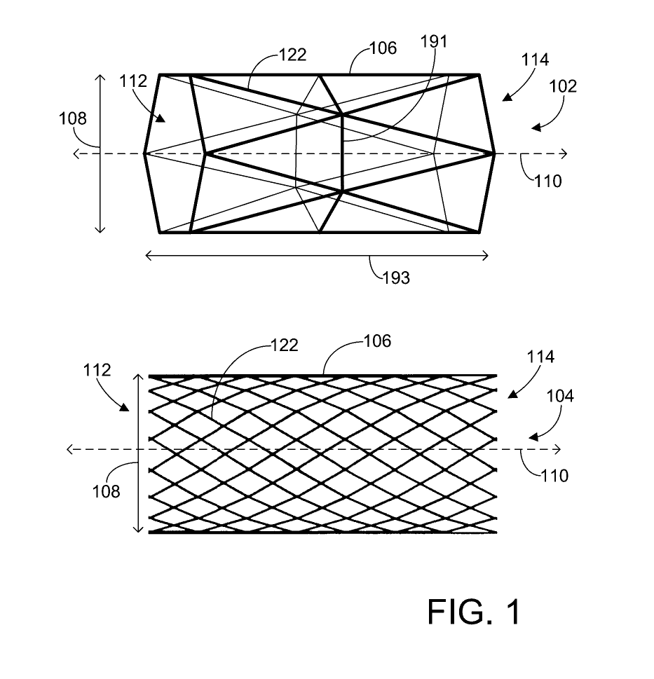

With reference to the figures, a stent 302 used in reducing a diameter of a transjugular intrahepatic portosystemic shunt 610 may comprise a tube-shaped framework 106 having a substantially constant diameter 108 along a longitudinal axis 110 of the framework 106 from a first open end 112 to a second open 114 end opposing the first open end. FIG. 1 shows example frameworks that may be included in a reducing stent in accordance with various embodiments. In particular, at 102, FIG. 1 shows an example framework 106 having a hexagonal cross-section along the longitudinal length of the framework. In this example, the framework comprises V-shaped wires or struts 122 that form the framework. For example, as shown at 102 in FIG. 1, a pair of struts may intersect at first ends of the struts and a leg (e.g., leg 191) may couple together the opposing ends of the pair of struts. For example, end 112 of the framework shown at 102 may comprise 12 struts with 6 legs; however, any suitable number of struts and/or legs in any suitable configuration may be used. As another example, at 104, FIG. 1 shows a framework 106 having a substantially circular cross-section along the longitudinal length of the framework. In this example, the framework is formed from interconnected wires or struts 122 that form a mesh having diamond-shaped perforations. It should be understood that the frameworks shown in FIG. 1 are given by way of example and any suitable tube-shaped framework having any suitable shape or configuration may be used without departing from the scope of this disclosure.

The tube-shaped framework 106 may be formed of any suitable material in any suitable configuration to form a structure having a tube-like shape having a substantially constant diameter along a longitudinal axis of the framework from a first open end to a second open end opposing the first open end. For example the struts 122 of framework 106 may be composed of a suitable metal, such as nitinol, to form a sleevelike part with perforated walls. The perforated walls of the framework may be honeycomblike or latticelike, for example. As an alternative, the framework may be woven, knit or knitted from various materials such as metals, plastics, etc.

In some examples, the framework 106 may collapsible such that the framework can be adjusted to a collapsed state wherein the diameter 108 is decreased. For example, if the framework is composed of nitinol, the framework may be pretreated so that in a low temperature position, it comprises a relatively stretched configuration of small diameter, so that it can be inserted by a catheter and placed in a body temperature position (high temperature position) in an enlarged form.

The length and diameter of the framework 106 may have any suitable dimensions. In one example embodiment, the cross-sectional diameter 108 of the framework may be approximately 10 mm. However, in other examples, the cross-sectional diameter 108 of the framework may be greater than or less than 10 mm. The framework 106 may have any suitable length 193. For example, the length 193 of the framework may be in a range of 1-10 cm.

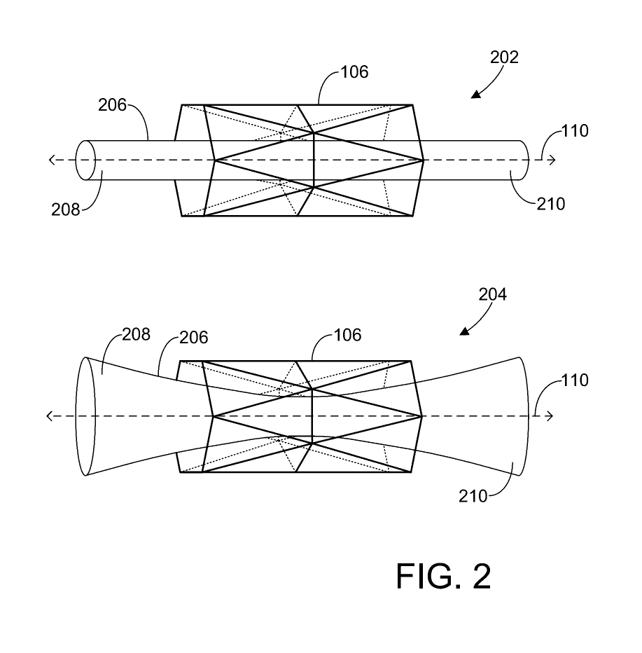

With reference to FIGS. 2-6, a reducing stent 302 used in reducing a diameter of a transjugular intrahepatic portosystemic shunt 610 comprises a tube-shaped framework 106 with a tubular material 206 affixed around an outer surface of the framework 106 and extending through the interior 314 of the framework 106 to define a reducing passage 322 in the interior of the framework.

The tubular material 206 may comprise any suitable expandable, biocompatible material. In some examples, the tubular material 206 may be composed of a material having one or more of the following properties: strength greater than a threshold (high strength-to-weight ratio), chemical inertness, biocompatibility, high thermal resistance (thermal resistance greater than a threshold), high chemical resistance in harsh environments, low flammability, low coefficient of friction, low dielectric constant, low water adsorption, and/or good weathering properties. For example, the tubular material may comprise a polytetrafluoroethylene-based material such as Teflon, expanded polytetrafluoroethylene (ePTFE), or other similar polymer.

As shown in the figures, the tubular material 206 has a first open end 208 and a second opposing open end 210. The first end 208 of the tubular material is affixed to a first region 304 around an outer surface of the framework 106 and the tubular material 206 extends from the first region 304 over an edge 312 of the first open end 112 of the framework 106, through an interior 314 of the framework 106, and over an edge 316 of the second open end 114 of the framework 106 to terminate at the second end 210 of the tubular material 206 which is affixed to a second region 306 around the outer surface of the tube-shaped framework 106 thereby defining a reducing passage 322 extending through the interior 314 of the tube-shaped framework 106 along the longitudinal axis 110 of the framework. A diameter 333 of the reducing passage 322 in a middle portion 336 of the interior 314 of the framework 106 is less than the diameters 108 of the framework at each of the first and second opposing ends of the framework.

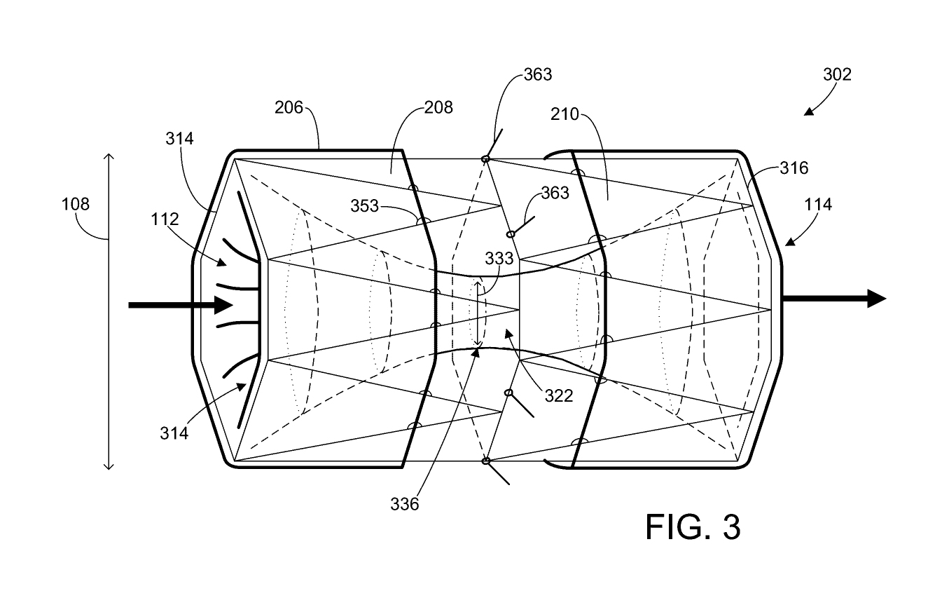

FIG. 2 illustrates a method of making a reducing stent 302 in accordance with various embodiments. At 202 in FIG. 2, tubular material 206 is shown after being inserted through the interior of framework 106. In view 202, the tubular material 106 has an elongated cylindrical shape with a first open end 208 and a second opposing open end 210. As shown at 204 in FIG. 2, the open ends, 208 and 210, of the tubular material may be expanded to increase the diameter at each open end so that the open ends of the tubular material can be wrapped around the edges 312 and 316 of the framework 106 to be affixed to an outer surface of the framework as shown in FIG. 3. The middle portion (the intraluminal center) 336 of the tubular material 106 may be unexpanded material (e.g., Teflon) defining a reducing passage 322 that tapers to the full stent track size at each end, 112 and 114, of the stent.

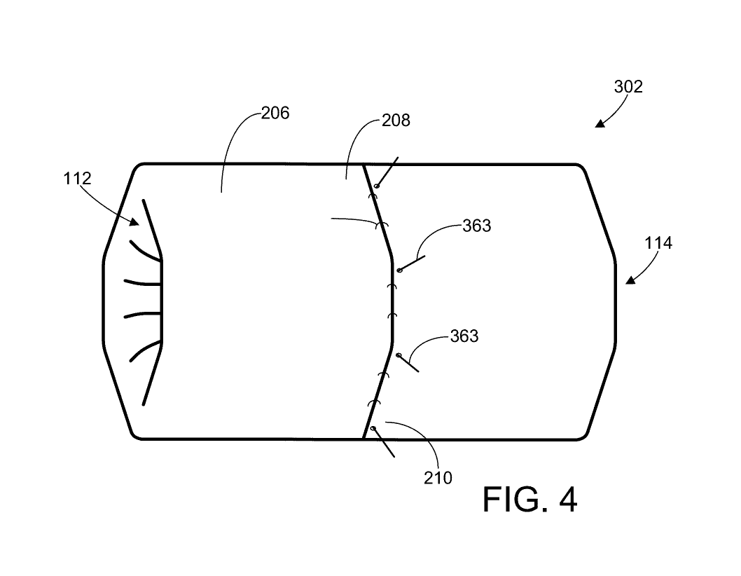

The tubular material may be coupled around the outer surface of the framework 106 in any suitable way. For example, the open ends 208 and 210 may be sutured to struts of the framework, e.g., via sutures 353 shown in FIG. 3 or 4, at the first and second regions, 304 and 306, around the outer surface of the framework. In some examples, as shown in FIG. 3, the first region 304, where end 208 of tubular material 206 is affixed, may not overlap with the second region 306, where end 210 of tubular material 206 is affixed, so that the there is a non-zero distance between the ends 208 and 210 of the tubular material 206 around the outer surface of the stent 302. However, in other examples, as shown in FIG. 4, the first region 304 where end 208 of tubular material 206 is affixed may overlap with or be adjacent to the second region 306 where end 210 of tubular material 206 is affixed so that the ends 208 and 210 of the tubular material 206 overlap or touch around the outer surface of the stent 302. For example, the tubular material may be wrapped around the stent ends to meet in approximately the middle on the outside surface of the stent.

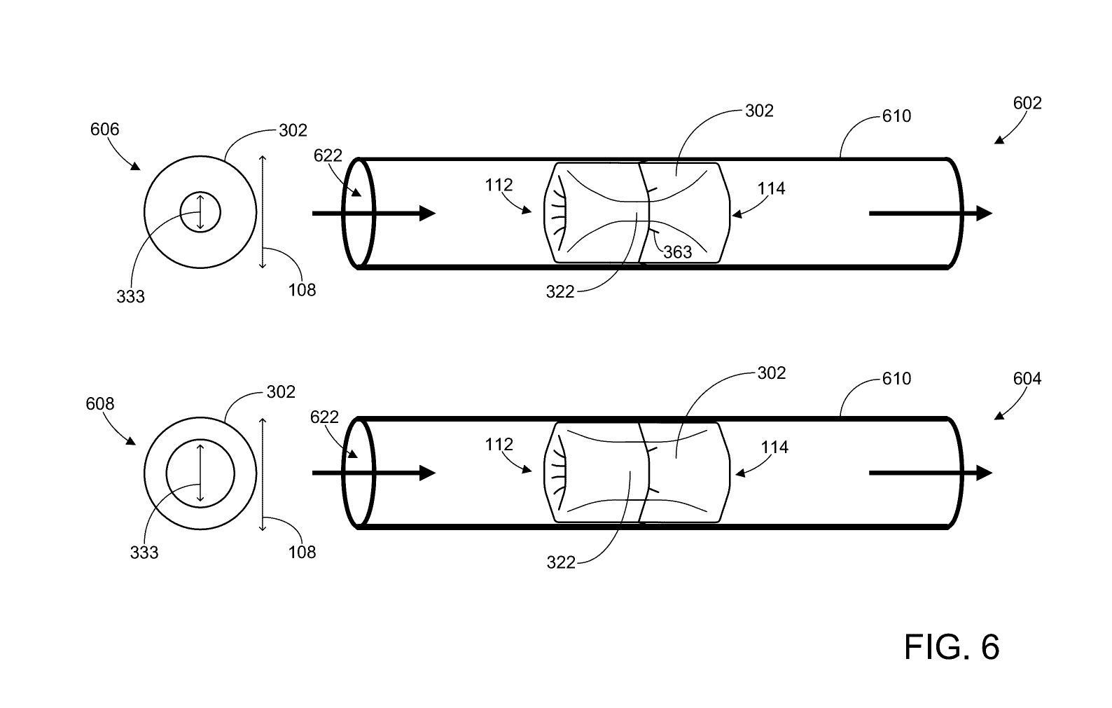

In some examples, stent 302 may further comprise a plurality of directional barbs 363 affixed to struts 122 of the framework 106 around the outer surface of the framework. For example, the directional barbs 363 may be affixed to struts around a central outer circumference of the stent. The directional barbs 363 may be affixed to the outer surface of the stent in any suitable may, e.g., via sutures. The barbs extend a non-zero distance above the outer surface of the stent and are angled away from an end of the stent, e.g., end 112. The directional barbs 363 confer a directionality to the stent 302 such that when the reducing stent 302 is placed in the interior of a shunt 610, e.g., as shown in FIG. 6, the stent 302 is oriented such that the barbs are angled in a direction opposing a flow of fluid (e.g., blood flow illustrated by the directional arrows in FIGS. 3 and 6) through the stent so that the reducing stent 302 is held in a fixed position within the shunt 610.

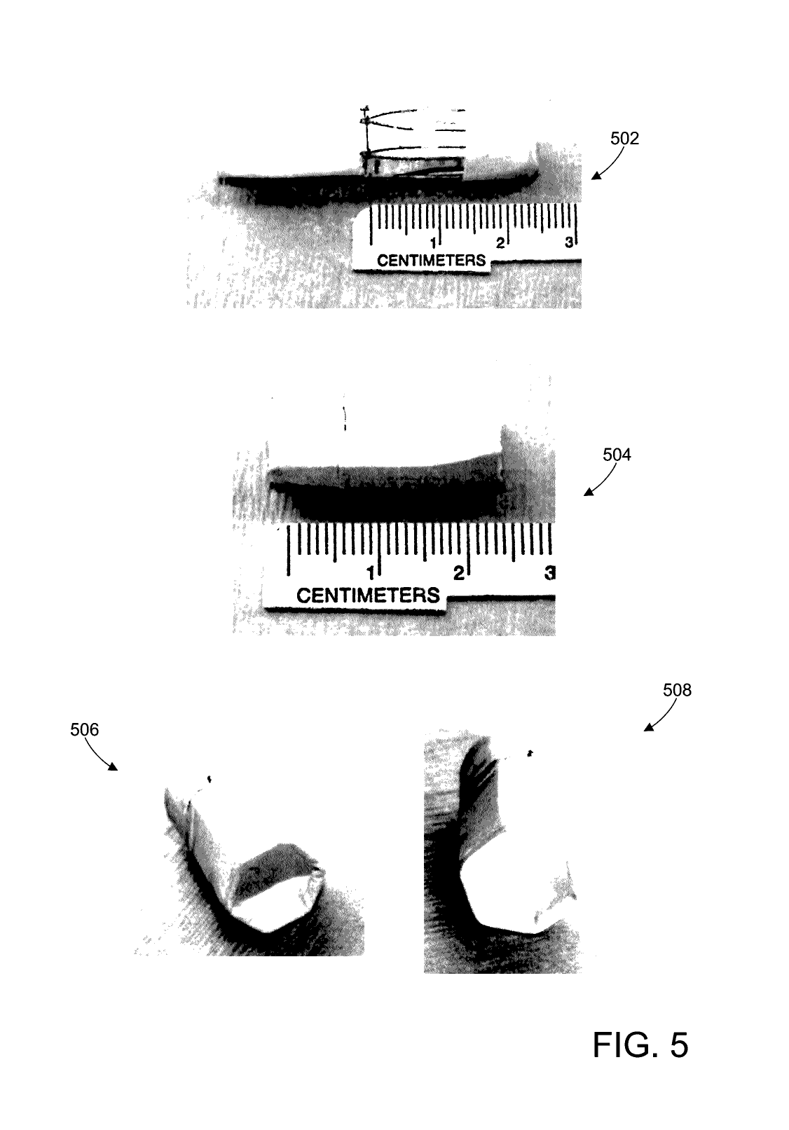

FIG. 5 shows additional example embodiments of a reducing stent in accordance with the disclosure. At 502, FIG. 5 shows a photograph of a side view of an example reducing stent. At 504, FIG. 5 shows a photograph of a side view of another example reducing stent having a length less than the example reducing stent shown at 502. At 506 and 508, FIG. 5 shows photographs of perspective views of example reducing stents in accordance with the disclosure.

FIG. 6 shows an example transjugular intrahepatic portosystemic shunt system in accordance with the disclosure. The transjugular intrahepatic portosystemic shunt system comprises a transjugular intrahepatic portosystemic shunt 610 and a reducing stent 302, examples of which are described above. The diameter 108 of reducing stent 302 may be approximately equal to or slightly less than a diameter of the cylindrical shunt 610 so that the reducing stent can be installed within an interior 622 of shunt 610.

The reducing passage 322 in reducing stent 322 comprises a pliable center piece formed from tubular material 206 that may be expanded if desired, e.g., after placement of the transjugular intrahepatic portosystemic shunt system. Thus, the diameter 333 of the reducing passage 322 in the middle portion 336 of the interior of the framework 106 may be adjustable from a first diameter to a second diameter larger than the first diameter. For example at 606 in FIG. 6 a cross-sectional view of stent 302 is shown where the diameter 333 of the reducing passage 322 in the stent is a first length; and, at 608, a cross-sectional view of stent 302 is shown where the diameter 333 of the reducing passage 322 in the stent is expanded, e.g., dilated by a balloon, to a second length greater than the first length. For example, the first diameter of the reducing passage 322 may be approximately 3 mm which could be expanded to a second diameter of approximately 5 mm.

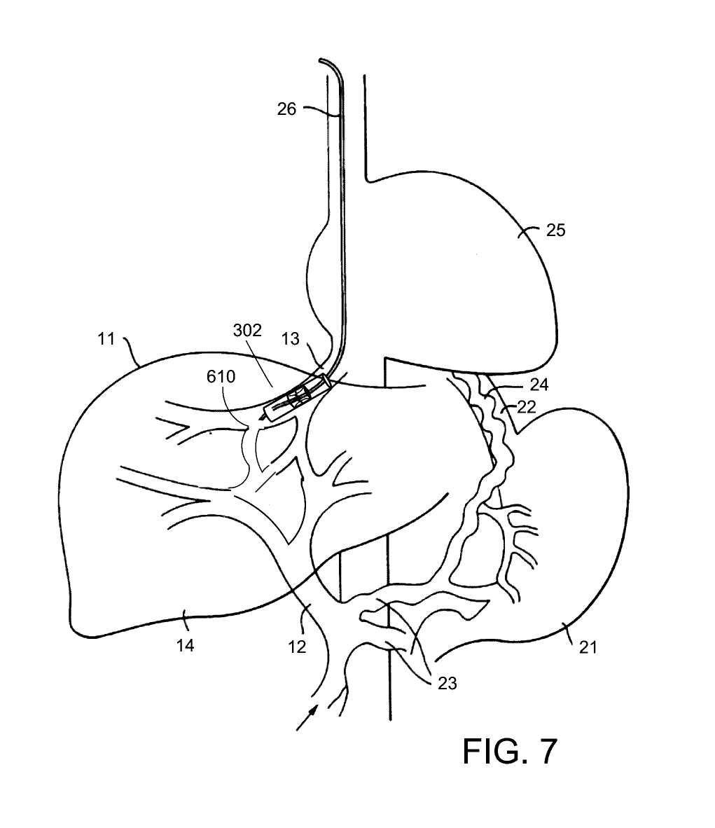

FIG. 7 shows an illustration of a shunt 610 including a reducing stent 302 installed therein between a portal vein and a hepatic vein in accordance with the disclosure. In particular, FIG. 7 diagrammatically illustrates a liver 11 with a portal vein 12 and a hepatic vein 13. Both veins usually branch into the hepatic tissue. It is discernible that blood vessels 23, leading from the portal vein to stomach 21 and to the esophagus branch off, which can form varices 24. Also, heart 25 and a feed catheter 26 pushed from the neck of the patient past heart 25 to a hepatic vein are shown.

In some examples, embodiments of the herein-described reducing stent may be used as follows. First, a shunt 610 may be provided between portal vein 12 and hepatic vein 13 by puncture by using a puncture needle. Into the shunt, the reducing stent 302 may be inserted by a catheter, e.g., by holding the shunt by a clamp or the like at the attaching site after insertion of the catheter containing the shunt and withdrawing the catheter, by which reducing stent 302 may be enlarged in its attached position as shown in FIG. 7, which, if it consists of a heat-treated nickel-titanium alloy (nitinol), is its high-temperature position. As alternative example, embodiments of the herein-described reducing stent may be used by first inserting the reducing stent 320 into shunt 610 and then delivering the shunt 610 with the reducing stent therein between portal vein 12 and hepatic vein 13. After placement of the shunt and the reducing stent therein, the reducing passage may be subsequently dilated, e.g., via a balloon or the like.

It is to be understood that the configurations and/or approaches described herein are exemplary in nature, and that these specific embodiments or examples are not to be considered in a limiting sense, because numerous variations are possible. The subject matter of the present disclosure includes all novel and nonobvious combinations and subcombinations of the various methods, systems, apparatuses, and configurations, and other features, functions, acts, and/or properties disclosed herein, as well as any and all equivalents thereof.

1. A stent used in reducing a diameter of a transjugular intrahepatic portosystemic shunt, the stent comprising:
a tube-shaped framework having a substantially constant diameter along a longitudinal axis of the framework from a first open end to a second open end opposing the first open end;
a tubular material with first and second opposing open ends, where the first end of the tubular material is affixed to a first region around an outer surface of the framework and wherein the tubular material extends from the first region over an edge of the first open end of the framework, through an interior of the framework, and over an edge of the second open end of the framework to terminate at the second end of the tubular material which is affixed to a second region around the outer surface of the tube-shaped framework thereby defining a reducing passage extending through the interior of the tube-shaped framework along the longitudinal axis of the framework, wherein a diameter of the reducing passage in a middle portion of the interior of the framework is less than the diameters of the framework at each of the first and second opposing ends of the framework.

2. The stent of claim 1, wherein the tubular material is sutured to one or more struts of the framework at the first and second regions around the outer surface of the framework.

3. The stent of claim 1, further comprising a plurality of directional barbs affixed to the struts of the framework around the outer surface of the framework.

4. The stent of claim 3, wherein the barbs are affixed to the struts around a central circumference of the stent.

5. The stent of claim 1, wherein the tubular material comprises an expandable, biocompatible material.

6. The stent of claim 5, wherein the tubular material comprises a polytetrafluoroethylene-based material.

7. The stent of claim 1, wherein the tube-shaped framework is collapsible.

8. The stent of claim 1, wherein the diameter of the reducing passage in the middle portion of the interior of the framework is adjustable from a first diameter to a second diameter larger than the first diameter.

9. The stent of claim 8, wherein the first diameter is approximately 3 mm.

10. The stent of claim 8, wherein the second diameter is approximately 5 mm.

11. The stent of claim 1, where the framework has a cross-sectional diameter of approximately 10 mm

12. The stent of claim 1, where the framework has a hexagonal cross-section.

13. The stent of claim 1, where the tube-shaped framework has a substantially circular cross-section.

14. The stent of claim 1, where the first region overlaps the second region.

15. The stent of claim 1, where the first region does not overlap the second region.

16. A transjugular intrahepatic portosystemic shunt system, comprising:
a transjugular intrahepatic portosystemic shunt; and
the stent of claim 1 mounted within an interior of the transjugular intrahepatic portosystemic shunt.
